(19) United States
(12) Patent Application Publication
(10) Pub. No.: US 2023/0301690 A1
(43) Pub. Date: Sep. 28, 2023

(54) REDUCER REMOVER

(71) Applicant: Astura Medical Inc., Irving, TX (US)

(72) Inventors: Ross Dusterhoft, Irving, TX (US); Thomas Purcell, Irving, TX (US)

(73) Assignee: Astura Medical Inc., Irving, TX (US)

(21) Appl. No.: 18/125,942

(22) Filed: Mar. 24, 2023

Related U.S. Application Data
(60) Provisional application No. 63/322,794, filed on Mar. 23, 2022.

(51) Int. Cl. A61B 17/70

(52) U.S. Cl. CPC A61B 17/7091; A61B 17/7086

(57) ABSTRACT

The present invention is directed to a reducer remover to uncouple or unlock a reducer from a tulip of a pedicle screw. To remove the reducer from the tulip, a reducer remover is inserted onto the reducer to engage and unlock the locking mechanism of the reducer. The reducer remover features a ram that extends distally and engages actuating members to move the actuating members outward. The outward movement of the actuating members engages the locking mechanism to disengage or unlock the reducer from the tulip. In one embodiment, the outward movement of the actuating members push on the reducer clips in an outward direction to disengage them from the tulip. If needed, the ram continues movement distally until contacting the rod or set screw within the tulip. This distal movement of the ram pushes the reducer off of the tulip, making the reducer easy to remove from the tulip.

### CROSS-REFERENCE TO RELATED APPLICATIONS

This application claims the benefit of U.S. Provisional Application No. 63/322,794 filed Mar. 23, 2023, which is incorporated herein by reference.

### FIELD

The present invention relates generally to the field of surgery, and more specifically, to a reducer remover for use in spinal fusion surgery.

### BACKGROUND

Many spinal fixation systems use pedicle screws attached to two or more vertebrae coupled to a fixation rod. The pedicle screw includes a body member or tulip that includes a tulip slot or U-shaped channel to accept the fixation rod. A set screw is used to threadably engage the body member of the screw assembly to secure the stabilizing rod within the body member. Positioning the spinal fixation rod in the screw head typically requires the drawing of the rod to the screw using a rod reducer.

Reducers are placed over the spinal fixation rod and attached to the pedicle screw body member or tulip. The reducer then pushes the spinal fixation rod into the tulip slot or U-shaped channel and a set screw is used to clamp the rod in place.

In certain situations, the reducers often become stuck on screw tulips due to proximity to the wound wall or interference with bony material. An instrument to assist in removing the reducer from the tulip is needed.

Thus, there is a need for instruments for removing stuck reducers that solves the problems listed above.

### SUMMARY

The present invention is directed to a reducer remover that is configured to remove a reducer that is stuck on the tulip of a spinal screw, such as a pedicle screw. The reducer remover features an actuator that is configured extend one or more actuating members outwardly to engage a locking mechanism on the reducer. When the actuating members extend outwardly, they are configured to push the locking mechanism outward away from the screw tulip to disengage the reducer from the screw tulip. If needed, the actuator continues distally toward the tulip until, contacting a rod or set screw within the tulip. The distal movement of the actuator pushes the reducer off of the tulip, making the reducer easy to remove from the tulip. The present application describes examples of actuation mechanisms, such as a ram, and is not limited by the examples as there are other mechanism may be used to drive the ram.

In some embodiments, the present invention relates to a method for removing a stuck reducer from a screw tulip using the above reducer remover. The method includes inserting the reducer remover into the reducer that is coupled to the tulip, moving actuating members outwardly to push on a reducer locking mechanism to disengage or unlock the reducer locking mechanism from the screw tulip. Once disengaged, the reducer is removed from the tulip.

### BRIEF DESCRIPTION OF THE DRAWINGS

FIG. 1 is a side view and FIG. 2 is a sectional view showing one embodiment of a reducer remover configured to couple with a reducer and unlock the reducer from the tulip of a pedicle screw.

FIG. 3 is a perspective view of the distal end of the reducer remover in FIG. 2 with the ram in a retracted state within the tubular body and the actuating teeth in a stored position.

FIG. 4 is a sectional side view of reducer remover showing initial movement of the ram with the actuating teeth pushed out.

FIG. 5 is a perspective end view of the distal end of the reducer remover in FIG. 4 showing initial movement of the ram with the actuating teeth pushed out.

FIG. 6 is a sectional side view of reducer remover showing the ram in the fully extended state.

FIG. 7 is a perspective end view of the distal end of the reducer remover in FIG. 7 showing the ram in the fully extended state.

FIG. 8 shows the ram in the retracted state within the tubular body.

FIG. 9 shows the ram moving distally with the distal portion extending from the distal end of the tubular body.

FIG. 10 shows the ram extending distally from the distal end of the tubular body in the fully extended state.

FIG. 11 shows a reducer having distal arms with clips coupled to clip recesses in a tulip of a pedicle screw.

FIG. 12 is a sectional side view showing the reducer remover coupled to the reducer and tulip of the pedicle screw.

FIG. 13 is a sectional side view showing the reducer remover coupled to the reducer and tulip of the pedicle screw with the ram in the fully extended state.

FIG. 14 is a side view and FIG. 15 is a sectional view showing another embodiment of a reducer remover having a twist handle configured to couple with a reducer and unlock the reducer from the tulip of a pedicle screw.

FIG. 16 is a perspective view of the distal end of the reducer remover in FIG. 15 with the ram in a retracted state within the tubular body and the actuating teeth in a stored position.

FIG. 17 is a sectional side view of reducer remover showing initial movement of the ram with the actuating teeth pushed out.

FIG. 18 is a perspective end view of the distal end of the reducer remover in FIG. 17 showing initial movement of the ram with the actuating teeth pushed out.

FIG. 19 is a sectional side view of reducer remover showing the ram in the fully extended state.

FIG. 20 is a perspective end view of the distal end of the reducer remover in FIG. 7 showing the ram in the fully extended state.

FIG. 21 is a side view and FIG. 22 is a sectional view showing another embodiment of a reducer remover having a lever handle configured to couple with a reducer and unlock the reducer from the tulip of a pedicle screw.

FIG. 23 is a perspective view of the distal end of the reducer remover in FIG. 22 with the ram in a retracted state within the tubular body and the actuating teeth in a stored position.

FIG. 24 is a sectional side view of reducer remover showing initial movement of the ram with the actuating teeth pushed out.

FIG. 25 is a perspective end view of the distal end of the reducer remover in FIG. 24 showing initial movement of the ram with the actuating teeth pushed out.

FIG. 26 is a sectional side view of reducer remover showing the ram in the fully extended state.

FIG. 27 is a perspective end view of the distal end of the reducer remover in FIG. 26 showing the ram in the fully extended state.

FIGS. 28A, 28B, 28C show different stages of movement by the ratcheting mechanism.

### DETAILED DESCRIPTION

A reducer is attached to a tulip of a pedicle screw during use. The reducer includes a locking mechanism, such as locking reducer clips, that engage recesses on the sides of the tulip. To remove the reducer from the tulip, a reducer remover is inserted onto the reducer to engage and unlock the locking mechanism of the reducer. The reducer remover features an actuator configured to move one or more actuating members outwardly from a stored position to a deployed position. The outward movement of the actuating members engages the locking mechanism to disengage or unlock the reducer from the tulip. In one embodiment, the outward movement of the actuating members push on the reducer clips in an outward direction to disengage them from the tulip. If needed, the actuator continues movement distally until contacting the rod or set screw within the tulip. This distal movement of the ram pushes the reducer off of the tulip, making the reducer easy to remove from the tulip.

In the examples shown below, the actuator includes a ram and ram actuator, and the actuating members include actuating teeth. The present application is not limited to the examples described herein; other mechanism and components may be used.

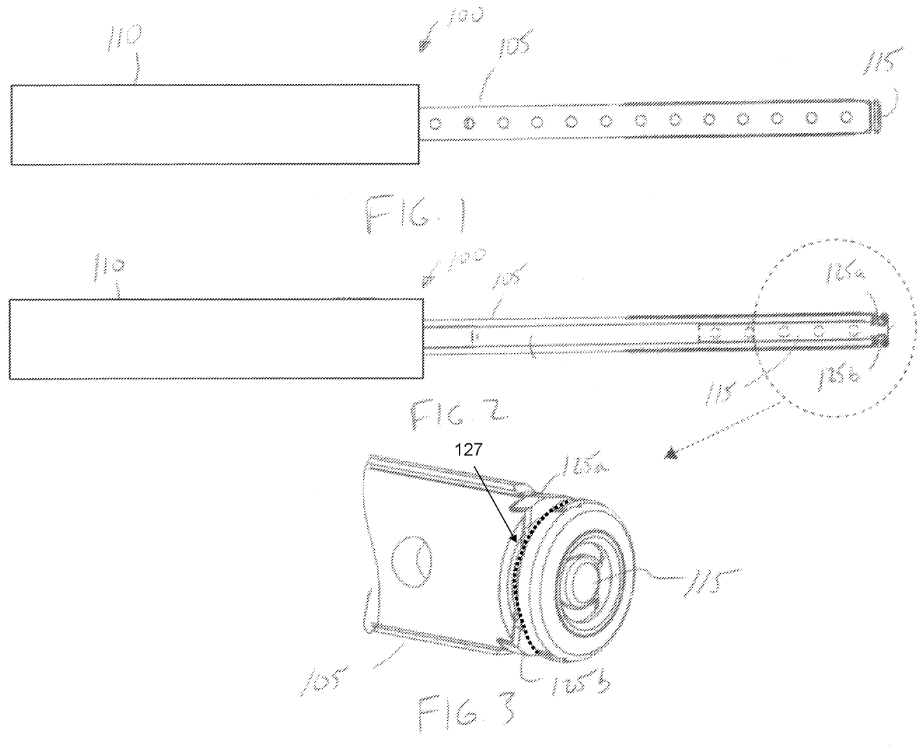

FIG. 1 is a side view and FIG. 2 is a sectional view showing one embodiment of a reducer remover 100 configured to couple with a reducer to uncouple or unlock the reducer from a tulip of a pedicle screw. The reducer remover includes a tubular body 105, a ram actuator 110, and a ram 115. The ram actuator 110 is positioned at a proximal end of the tubular body 105 and the ram 115 is positioned within the tubular body 105. The ram actuator 110 is coupled to a proximal end of the ram 115. In use, the ram actuator 110 is configured to move the ram 115 distally inside the tubular body 105.

The ram actuator 110 may include many types of devices that can advance the ram 115 distally, including manually operated and electrically powered. In some embodiments the ram actuator 110 may be a twist handle with a threaded ram, so when the handle is twisted, the threaded ram advances distally. In some embodiments the ram actuator 110 may be a lever handle that when squeezed pushes the ram distally. In some embodiments the ram actuator may be an impact driver, either manual or battery powered, that hits the ram distally. In some cases, the ram actuator may be a screwdriver, either manual or battery powered, that rotates a threaded ram distally.

Two actuating teeth 125a, 125b are positioned at the distal end of the tubular body 105. The ram 115 includes a distal portion 130, a ramped or conical portion 135, and a proximal portion 140. The two actuating teeth 125a, 125b are configured to slidingly couple with the ram 115. As the ram 115 is moved distally, the two actuating teeth 125a, 125b slide along the distal portion 130, the ramped or conical portion 135, and proximal portion 140 the of the ram 115 and push the two actuating teeth 125a, 125b outwardly from a stored position to a deployed position. When the two actuating teeth 125a, 125b are in contact with the distal portion 130 of the ram 115 they are in the stored position. When the two actuating teeth 125a, 125b are in contact with the ramped or conical portion 135 of the ram 115, they move outwardly from the stored position to the deployed position. When the two actuating teeth 125a, 125b are in contact with the proximal portion 140 of the ram 115 they stay in the deployed position.

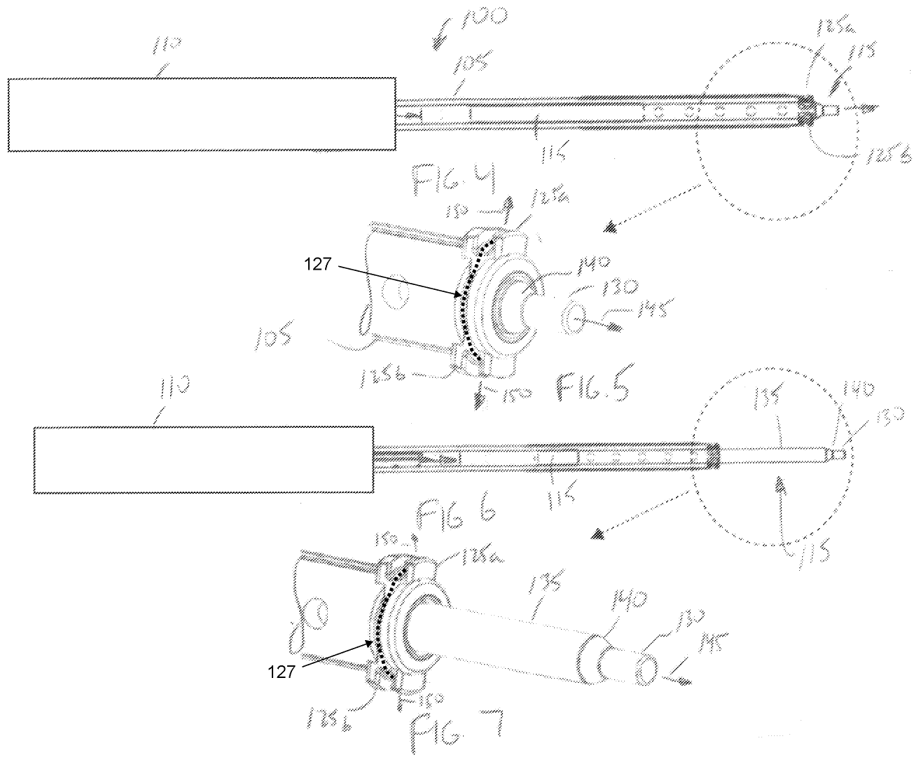

The actuating teeth 125a, 125b are held in position with an actuating teeth retainer 127 positioned within a slot in the tubular body 105 and a groove in the actuating teeth 125a, 125b (dashed line in FIGS. 3, 5, 7). The actuating teeth retainer 127 is made of a flexible material, such as a spring or elastic material, that is biased to hold the actuating teeth 125a, 125b in the stored position during insertion and removal of the reducer, and also can stretch as the actuating teeth 125a, 125b move outward from the stored position (FIG. 3) to the deployed position (FIGS. 5, 7).

The present application describes examples of reducer remover 100 having two actuating teeth 125a, 125b but the invention is not limited by the examples as there are other number of actuating teeth may be used to unlock the reducer. The actuating teeth may have different shapes to engage various shapes of the locking mechanism or clips on the reducers.

FIG. 3 is a perspective view of the distal end of the reducer remover 100 with the ram 115 positioned within the tubular body 105 and the actuating teeth 125a, 125b in a stored position. In the stored position, the actuating teeth 125a, 125b are in contact with the distal portion 130 of the ram 115.

FIGS. 4-7 show various stages of the ram 115 moving distally and the distal end extending from the distal end of the tubular body 105. The figures show the ram 115 having the multiple portions with differing diameters and profiles, including the distal portion 130 having a first diameter, the ramped or conical portion 135 that transitions from the first diameter to a second diameter, and the proximal portion 140 having the second diameter, with the second diameter being larger than the first diameter. As pointed out above, when the ram 115 is moved distally, the two actuating teeth 125a, 125b slide along the multiple portions of the ram 115 from the first diameter to the second diameter, which moves them outwardly from the stored position to the deployed position.

FIG. 4 is a sectional side view of reducer remover 100 and FIG. 5 is a perspective end view showing initial movement of the ram 115 from the distal end of the tubular body 105. As the ram 115 is moved distally, the distal end of the ram 115 extends from the tubular body 105 and the ramped or conical portion 135 engages the actuating teeth 125a, 125b and moves them in an outward direction 150 as they slide up the ramped or conical portion 135.

FIG. 6 is a sectional side view of reducer remover 100 and FIG. 7 is a perspective end view showing the ram 115 in the fully extended state. The ram actuator 110 moves the ram 115 distally 145 to fully extend from the tubular body 105. When the ram 115 is fully extended distally 145, the proximal portion 140 engages the actuating teeth 125a, 125b and fully extends them in the outward direction 150.

Actuating Teeth Deployment

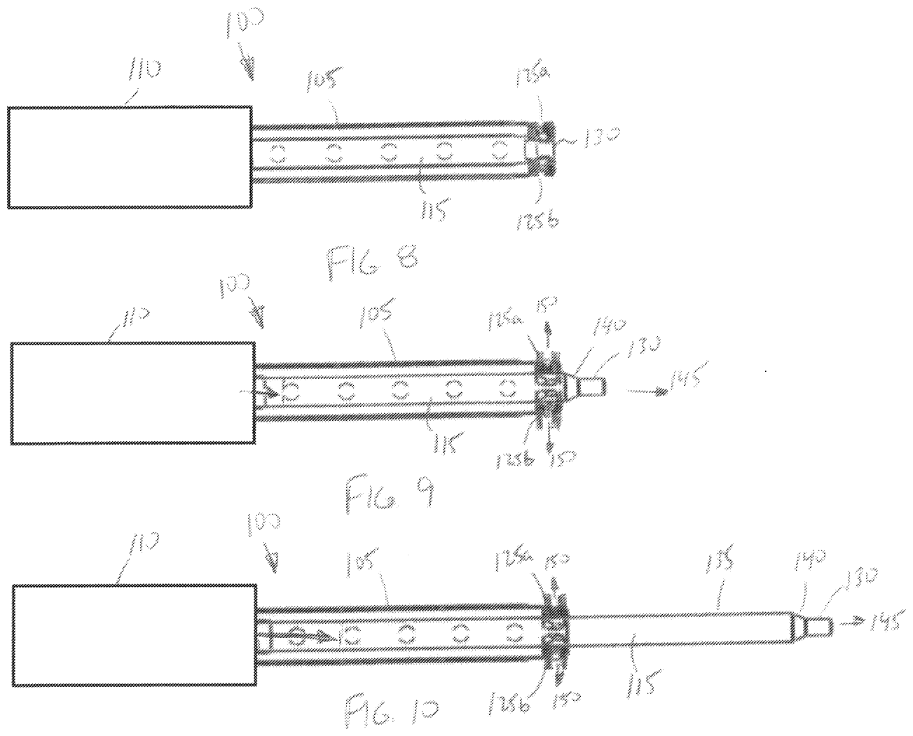

FIGS. 8-10 show the different stages of deployment of the actuating teeth 125a, 125b by the ram 115.

FIG. 8 shows the ram 115 in the retracted state within the tubular body 105. In the retracted state, the actuating teeth 125a, 125b are in the stored position. Also in the retracted state, actuating teeth 125a, 125 are coupled to the distal portion 130. The first diameter of the distal portion 130 is configured not to move the actuating teeth 125a, 125b outward from the stored position.

FIG. 9 shows the ram 115 moving distally 145 with the distal portion 130 extending from the distal end of the tubular body 105. As the ram 115 moves distally, the actuating teeth 125a, 125b slides up the ramped or conical portion 135 from the first diameter to the second diameter, which pushes them in an outward direction 150, from the stored position to the deployed position.

FIG. 10 shows the ram 115 extending distally 145 from the distal end of the tubular body 105 in the fully extended state. The actuating teeth 125a, 125b are in contact with the proximal portion 140 keeping them in the deployed position.

Actuating Teeth Engaging Clips for Removal

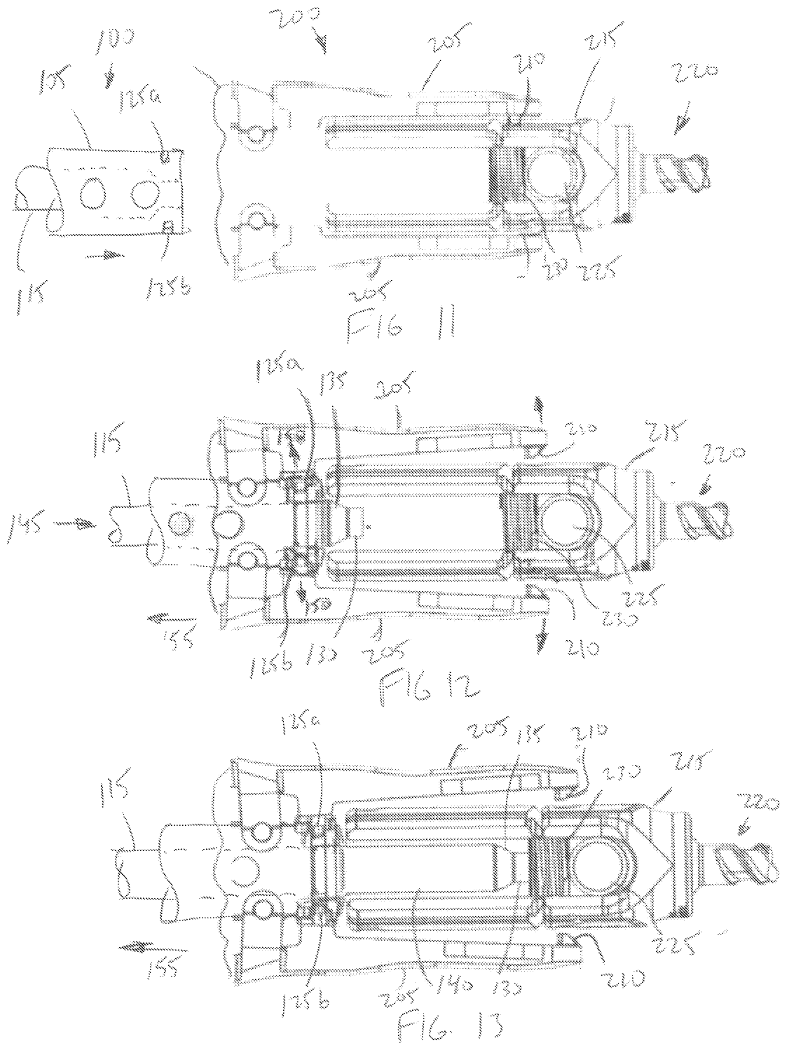

FIG. 11 shows one example of the distal portion of a reducer 200 coupled to a tulip 215 of a pedicle screw 220 after reduction of a spinal rod 225 into a U-shaped portion of the tulip 225. The spinal rod 225 is held in place by a set screw 230. In this example, the reducer 200 includes distal arms 205 with clips 210 that are configured to couple to clip recesses in a tulip 215 of a pedicle screw 220.

The reducer 200 may become stuck on the tulip 215 due to proximity to the wound wall or interference with bony material. The present invention is directed to a reducer remover 100 that is designed to remove the reducer 200 from the tulip 215.

FIG. 12 is an enlarged section view showing the reducer remover 100 coupled to a reducer 200 and a tulip 215 of the pedicle screw 220. The reducer remover 100 clips onto the reducer 200 and aligns itself with the tulip 215. The ram actuator 110 advances the ram 115 distally 145, with the ramped or conical portion 135 engaging the two actuating teeth 125a, 125b and extending them outwardly 150. The actuating teeth 125a, 125b are configured to push on the distal arms 205 of the reducer 200 outward 235 and disengage the clips 210 them from the screw tulip 215.

The ram actuator 110 continues to advance the ram 115 distally 145 to fully extend from the tubular body 105. When the ram 115 is fully extended distally 145, the proximal portion 140 engages the actuating teeth 125a, 125b and so they are extended in the outward direction 150 in the deployed position. Once disengaged, the reducer 200 is removed 155 from the tulip 215.

In some cases, the reducer 200 may need additional help in removal from the tulip 215.

FIG. 13 is an enlarged section view showing the reducer remover 100 providing additional help to remove the reducer 200 from the tulip 215. In this situation, the ram actuator 110 continues to move the ram 115 distally until the distal end contacts the spinal rod 225 or set screw 230 within the tulip 215. Continued movement of the ram 115 distally to the fully extended state pushes 155 the reducer 200 off of the tulip 215, making the reducer 200 easy to remove from the tulip 215 of the pedicle screw 220.

Reducer Remover with Twist Handle

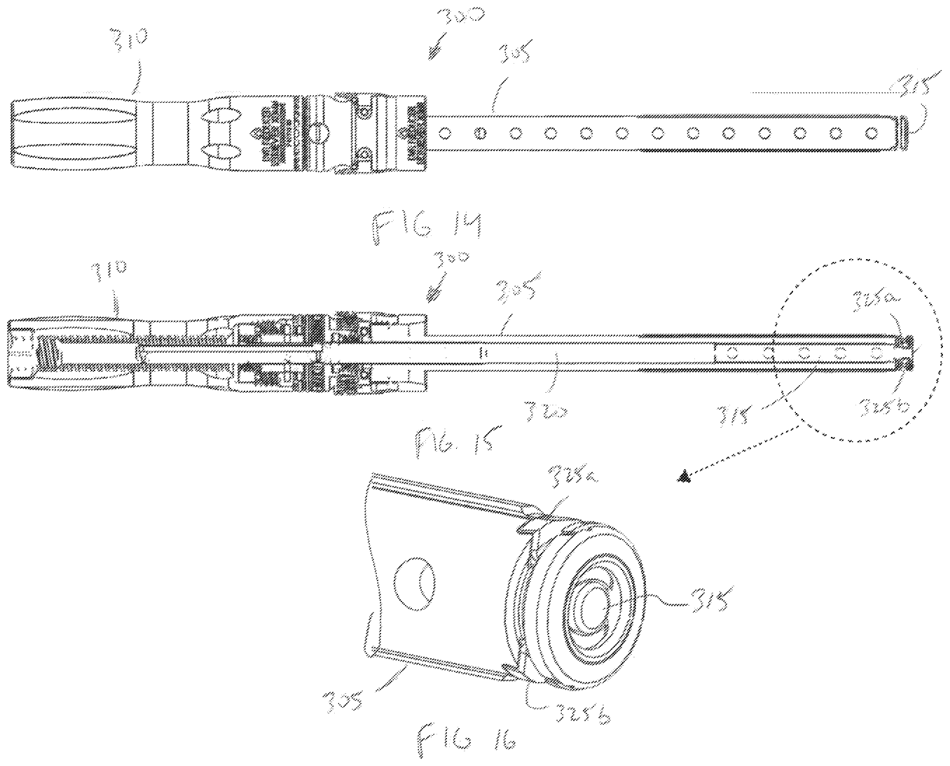

FIG. 14 is a side view and FIG. 15 is a sectional view showing one embodiment of a reducer remover 300 with a twist handle 310 for actuation of a threaded ram 315. The reducer remover 300 is configured to couple with a reducer to uncouple or unlock the reducer from a tulip of a pedicle screw. The reducer remover includes a tubular body 305, a twist handle 310, a threaded ram 315, and a driver shaft 320. The twist handle 310 is positioned at a proximal end of the tubular body 305 and the threaded ram 315 and driver shaft 320 are positioned within the tubular body 305. The driver shaft 320 includes a proximal end coupled to the twist handle 310 and a distal end coupled to the threaded ram 315. In use, rotation of the twist handle 310 moves the driver shaft 320 and extends the threaded ram 315 from a distal end of the tubular body 305.

Two actuating teeth 325a, 325b are positioned at the distal end of the tubular body 305. The two actuating teeth 325a, 325b are slidingly coupled to the threaded ram 315. The threaded ram 315 includes a distal portion 330, a ramped or conical portion 335, and a proximal portion 140 joining the proximal and distal portions. 330, 335. As the twist handle 310 is rotated the threaded ram 315 is moved distally and the two actuating teeth 325a, 325b slide along the different portions of the treaded ram 315 and move outwardly from a stored position to a deployed position. When the two actuating teeth 325a, 325b are in contact with the distal portion 330 they are in the stored position. When the two actuating teeth 325a, 325b are in contact with the ramped or conical portion 340, they move outwardly from the stored position to the deployed position. When the two actuating teeth 325a, 325b are in contact with the proximal portion 335 they are in the fully deployed position.

FIG. 16 is a perspective view of the distal end of the reducer remover 300 with the threaded ram 315 in a retracted state within the tubular body 305 and the actuating teeth 325a, 325b in the stored position. In the stored position, the actuating teeth 125a, 125b are in contact with the distal portion 130 of the threaded ram 115.

FIGS. 17-23 show various stages of the threaded ram 315 extending from the distal end of the tubular body 305. The figures show that the threaded ram 315 includes multiple portions having different diameters, including a distal portion 330 having a first diameter, a ramped or conical portion 335 transitioning from the first diameter to a second diameter, and a proximal portion 340 having a second diameter. The second diameter being larger than the first diameter. As the twist handle 310 is rotated 355, the threaded ram 315 moves distally and the two actuating teeth 325a, 325b slide along the multiple portions of the treaded ram 315 and move outwardly from the stored position to the deployed position.

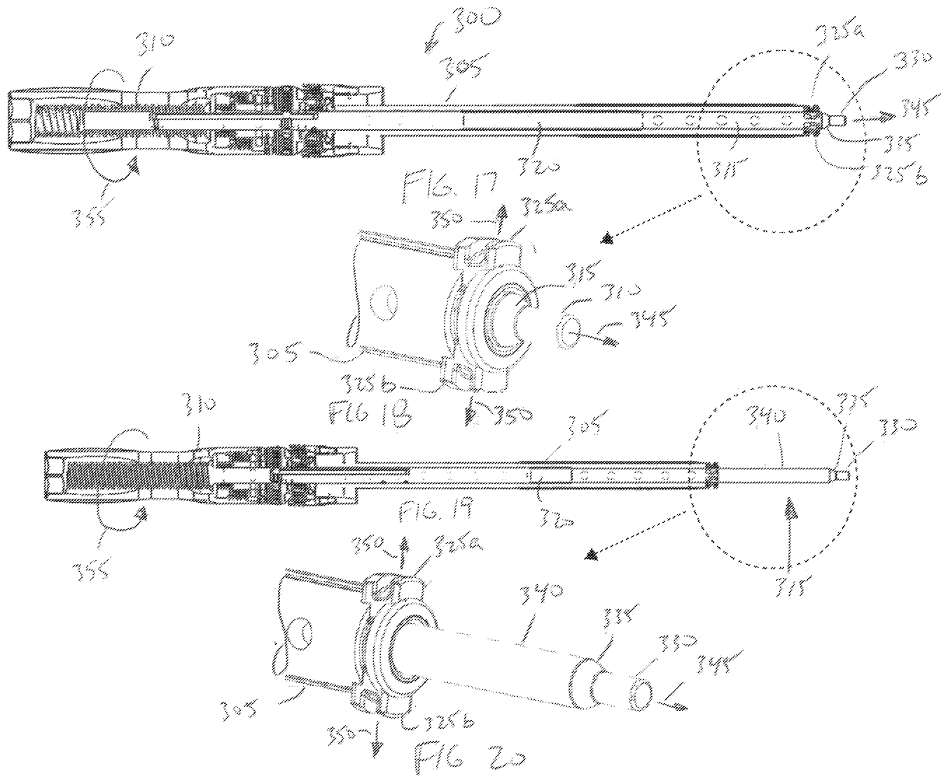

FIG. 17 is a sectional side view of reducer remover 300 and FIG. 18 is a perspective end view showing initial movement of the threaded ram 315 from the distal end of the tubular body 305. When the twist handle 310 is rotated 355, the drive shaft 320 is configured to move the threaded ram 315 distally 345 to extend from the tubular body 305. When the threaded ram 315 is extended distally 345, the actuating teeth 325a, 325b slide up the ramped or conical portion 340 and moves them in an outward direction 150.

FIG. 19 is a sectional side view of reducer remover 300 and FIG. 20 is a perspective end view showing the threaded ram 315 in the fully extended state. The twist handle 310 continues to rotate and the drive shaft 320 moves the threaded ram 315 distally 345 to fully extend from the tubular body 305. When the threaded ram 315 is fully extended distally 345, the proximal portion 335 engages the actuating teeth 325a, 325b and so they are fully extended in the outward direction 350.

Reducer Remover with Lever Handle

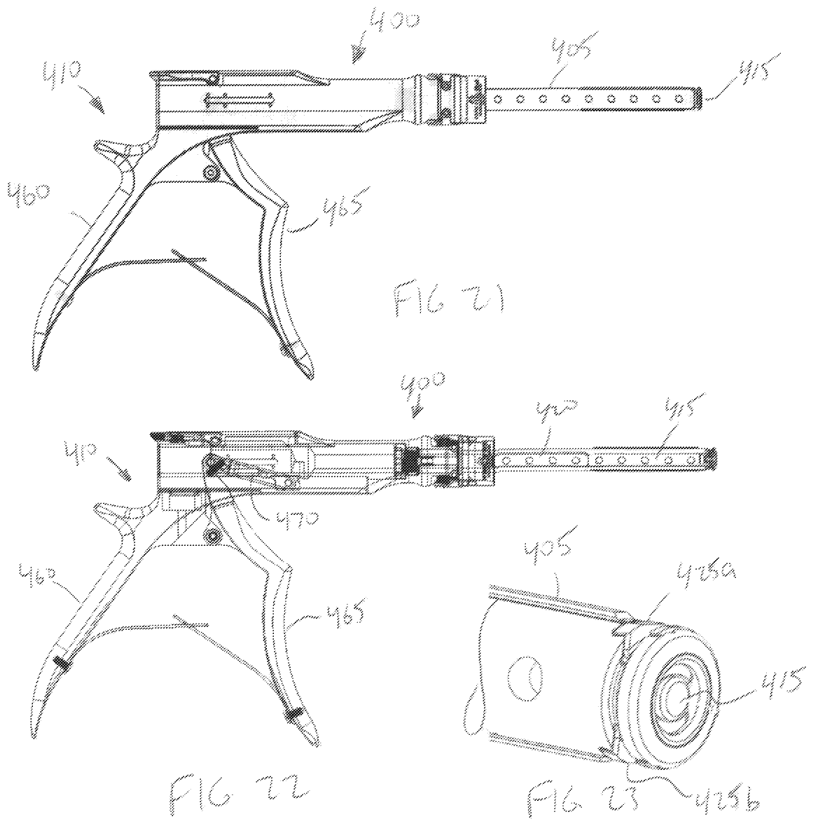

FIG. 21 is a side view and FIG. 22 is a sectional view showing another embodiment of a reducer remover 400 configured to couple with a reducer to uncouple or unlock the reducer from a tulip of a pedicle screw. The reducer remover 400 includes a tubular body 405, a lever handle 410, a ram 415, and a driver shaft 420. The tubular body 405 is coupled to the distal end of the lever handle 410, and the ram 415 and driver shaft 420 are positioned within the tubular body 405.

The lever handle 410 includes a fixed lever 460 pivotally connected with a hand operated lever 465. The hand operated lever 465 is coupled to linkage 470 that is configured to push the driver shaft 420 and the ram 415 distally when the hand operated lever 465 is pulled toward the fixed lever 460. The linkage 470 includes a ratcheting tooth that keeps the rod in three possible positions: retracted, actuating teeth pushed out, and fully extended.

Two actuating teeth 425a, 425b are positioned at the distal end of the tubular body 405. The two actuating teeth 425a, 425b are slidingly coupled to the ram 415. The ram 415 includes a distal portion proximal portion 430, a ramped or conical portion 435, and a proximal portion 440. As the ram 415 is moved distally, the two actuating teeth 425a, 425b slide along the portions of the ram 415 and move outwardly from a stored position to a deployed position. When the two actuating teeth 425a, 425b are in contact with the distal portion 430 they are in the stored position. When the two actuating teeth 425a, 425b are in contact with the ramped or conical portion 435, they move outwardly from the stored position to the deployed position. When the two actuating teeth 425a, 425b are in contact with the proximal portion 430 they are in the fully deployed position.

FIG. 23 is a perspective view of the distal end of the reducer remover 400 with the ram 415 in a retracted state within the tubular body 405 and the actuating teeth 425a, 425b in the stored position. In the stored position, the actuating teeth 425a, 425b are in contact with the distal portion 430 of the ram 415.

FIGS. 24-27 show various stages of the ram 415 extending from the distal end of the tubular body 405. The figures show that the ram 415 includes multiple portions having different diameters, including a distal portion 430 having a first diameter, a ramped or conical portion 340 transitioning from the first diameter to s second diameter. The second diameter being larger than the first diameter. As pointed out above, as ram 415 is moved distally, the two actuating teeth 425a, 425b slide along the portions of the ram 415 and move outwardly from the stored position to the deployed position.

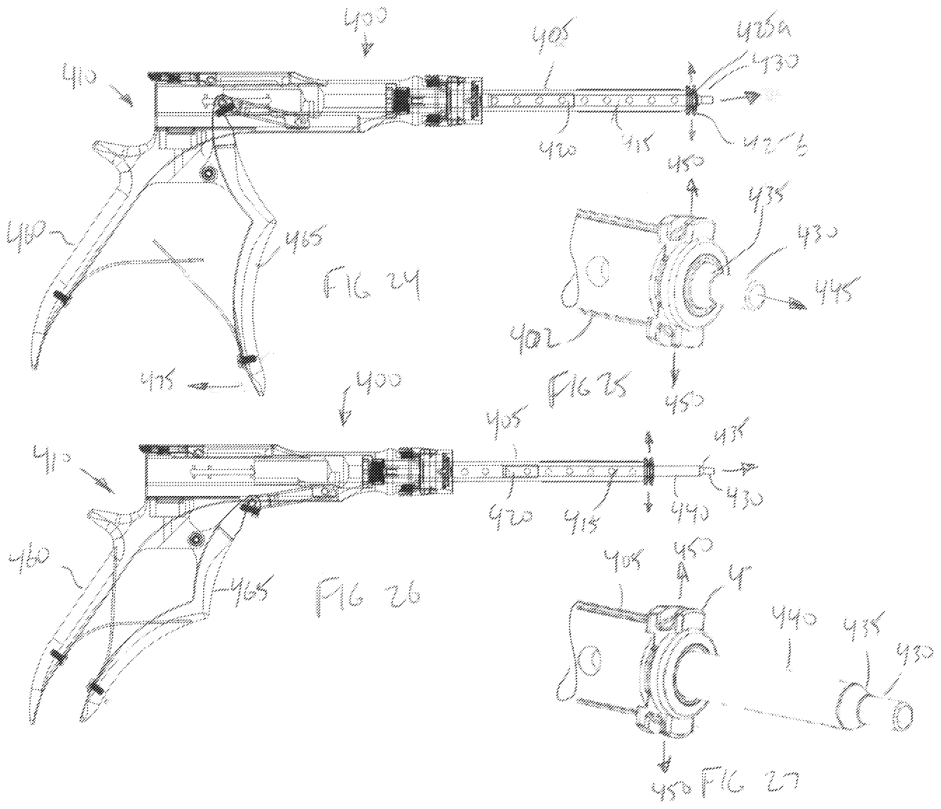

FIG. 24 is a sectional side view of the reducer remover 400 and FIG. 25 is a perspective end view showing initial movement of the ram 415 from the distal end of the tubular body 405. As the ram 415 is moved distally, the ramped or conical portion 440 engages the actuating teeth 425a, 425b and moves them outwardly 450 from the stored position to the deployed position. When the hand operated lever 465 has been moved 475 toward the fixed lever 460 and the linkage 470 has been ratcheted, pushing the driver shaft 420 and the ram 415 distally to the actuating teeth pushed out position. When the ram 415 is extended distally 445, the ramped or conical portion 435 engages the actuating teeth 425a, 425b and moves them in an outward direction 450 as they slide up the ramped or conical portion 440.

FIG. 26 is a sectional side view of reducer remover 400 and FIG. 27 is a perspective end view showing the ram 415 in the fully extended state. The hand operated lever 465 continues to move 475 toward the fixed lever 460 and the linkage 470 has been ratcheted so the ram 415 is fully extended. When the ram 415 is fully extended distally 445, the proximal portion 440 engages the actuating teeth 425a, 425b and so they are fully extended in the outward direction 450.

Lever Handle with Ratcheting Mechanism

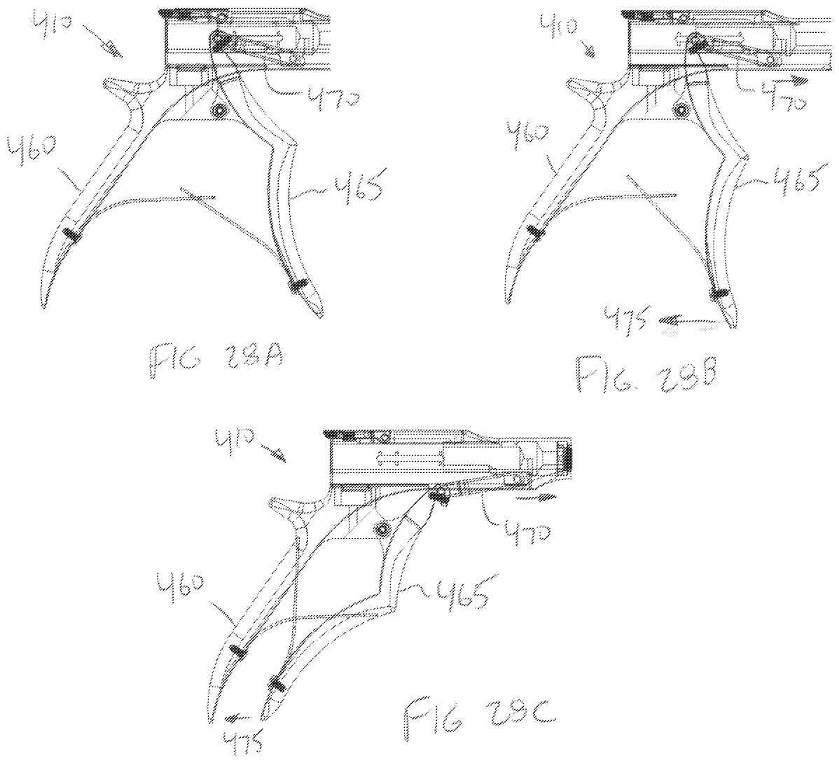

FIGS. 28A-28C show enlarged cross-sectional views of the ratcheting mechanism 470. The ratcheting mechanism 470 is coupled to the hand operated lever 465, and movement of the hand operated lever 465 toward the fixed lever 460 moves the ratcheting mechanism to different positions, starting with a retracted position, then to an actuating position, and finally to a ram fully extended position. In the retracted position, the actuating teeth 425a, 425b may engage the distal end 430 of the ram 415. In the actuating teeth pushed out position, the ratcheting mechanism 470 pushed the ram 415 distally and the actuating teeth 425a, 425b slide up the ramped or conical portion 435 and are pushed outward 450. In the fully extended position, the ratcheting mechanism 470 continues to push the ram 415 distally until it is fully extended.

FIG. 28A shows the handle 410 with the hand operated lever 465 in the first position with the actuating teeth 425a, 425b in the retracted position. This is the initial position for the delivery of the reducer remover 400.

FIG. 28B shows the handle 410 with the hand operated lever 465 in the second position with the actuating teeth 425a, 425b pushed out. In this position, the hand operated lever 465 has been moved 475 toward the fixed lever 460 and the linkage 470 has been ratcheted, pushing the driver shaft 420 and the ram 415 distally to the pushed out position for the actuating teeth 425a, 425b.

FIG. 28C shows the handle 410 with the hand operated lever 465 in the third position with the ram 415 in the fully extended position. In this position, the hand operated lever 465 continues to move 475 toward the fixed lever 460 and the linkage 470 has been ratcheted so the ram 415 is fully extended. When the ram 415 is being fully extended it may contact the spinal rod 225 or set screw 230 within the tulip 215 and push the reducer 200 off of the tulip 215, making the reducer 200 easy to remove from the tulip 215 of the pedicle screw 220.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A device for removing a reducer from a spinal screw comprising:
one or more actuating members configured to engage a locking mechanism of the reducer; and
an actuator configured to move the one or more actuating members outwardly from a stored position to a deployed position;
wherein the outward movement of the one or more actuating members is configured to unlock the locking mechanism from the spinal screw.

2. The device of claim 1, wherein outward movement of the one or more actuating members is configured to push the locking mechanism outward from the spinal screw to disengage the reducer.

3. The device of claim 1, wherein the actuator includes a tapered distal end slidably coupled to the one or more actuating members, wherein during movement of the actuator, the one or more actuating members are configured to slide along the tapered distal end to move outwardly from the stored position to the deployed position.

4. The reducer remover of claim 3, wherein the distal tapered end includes a distal portion having a first diameter, a ramped or conical portion that transitions from the first diameter to a second diameter, and a proximal portion having the second diameter, the second diameter being larger than the first diameter.

5. The reducer remover of claim 4, wherein when the one or more actuating members are in contact with the distal portion they are in the stored position, when the one or more actuating members are in contact with the ramped or conical portion they move outwardly from the stored position to the deployed position, and when the one or more actuating members are in contact with the proximal portion they stay in the deployed position.

6. The reducer remover of claim 1, further comprising a tubular body configured for insertion in a reducer, the one or more actuating members coupled to the tubular body and the actuator slidably positioned in the tubular body, wherein during distal movement of the actuator in the tubular body engages the one or more actuating members to move them outwardly from the stored position to the deployed position.

7. The device of claim 1, wherein actuator is further configured to push the reducer off of the spinal screw.

8. A device for removing a reducer from a spinal screw comprising:
one or more actuating members configured to engage a locking mechanism of the reducer; and
an actuator having a tapered distal end slidably coupled to the one or more actuating members, the tapered distal end configured to engage the one or more actuating members to move them outwardly from a stored position to a deployed position;
wherein the outward movement of the one or more members is configured to move the locking mechanism outward to disengage or unlock the locking mechanism from the spinal screw.

9. The reducer remover of claim 1, further comprising a tubular body configured for insertion in a reducer, the one or more actuating members coupled to the tubular body and the actuator slidably positioned in the tubular body, wherein during distal movement of the actuator in the tubular body engages the one or more actuating members to move them outwardly from the stored position to the deployed position.

10. The reducer remover of claim 1, wherein the distal tapered end includes a distal portion having a first diameter, a ramped or conical portion that transitions from the first diameter to a second diameter, and a proximal portion having the second diameter, the second diameter being larger than the first diameter.

11. The reducer remover of claim 3, wherein when the one or more actuating members are in contact with the distal portion they are in the stored position, when the one or more actuating members are in contact with the ramped or conical portion they move outwardly from the stored position to the deployed position, and when the one or more actuating members are in contact with the proximal portion they stay in the deployed position.

12. A device for removing a reducer from a spinal screw comprising:
a body with one or more actuating members configured for insertion in a reducer having a locking mechanism; and
an actuator slidably positioned in the tubular body, the actuator having a tapered distal end configured to engage the one or more actuating members to move them outwardly from a stored position to a deployed position to engage the locking mechanism of the reducer.
wherein the outward movement of the one or more members is configured to move the locking mechanism outward to disengage or unlock the locking mechanism from a spinal screw.
